(19) United States
(12) Patent Application Publication
(10) Pub. No.: US 2005/0070864 A1
(43) Pub. Date: Mar. 31, 2005

(54) Disposable waste receptacle

(76) Inventor: Leslie Fellion, Racine, WI (US)

Correspondence Address: JANSSON, SHUPE & MUNGER, LTD, 245 MAIN STREET, RACINE, WI, 53403, US

(21) Appl. No.: 10/850,166

(22) Filed: May 20, 2004

Related U.S. Application Data
(60) Provisional application No. 60/505,519, filed on Sep. 25, 2003.

(51) Int. Cl. A61F 5/44

(52) U.S. Cl. 604/346

(57) ABSTRACT

A disposable device and method for receiving and disposing waste is disclosed. The device comprises a bag and a foldable funnel, the bag defining a cavity and having an aperture and the funnel having a first opening and a second opening, the funnel bonded to the bag at the aperture such that the second opening is bounded by the bag, the funnel including a sealing element to seal the funnel to enclose the cavity. The method includes providing such a device, inserting waste into the bag, and folding the funnel such that the waste in enclosed within the cavity.

### CROSS-REFERENCE TO RELATED APPLICATION

This application claims benefit under 35 USC § 119(e) to U.S. Provisional Application No. 60/505,519, filed Sep. 25, 2003, the entire disclosure of which is incorporated herein by reference.

### FIELD OF THE INVENTION

The invention relates generally to receptacles for receiving waste, and, more particularly, to such receptacles which are intended to be disposed of after receiving such waste.

### BACKGROUND OF THE INVENTION

Often, during medical observation, the volume of fluids entering and exiting the body of a sick patient must be collected and monitored. Such collection and volume measurement may be done to evaluate the patient's progress toward better health or to assure that the patient maintains an acceptable fluid balance between intake and discharge.

Many illnesses result in frequent vomiting by the patient in which a quantity of fluid is discharged from the body. The complete control and measurement of bodily fluid balance requires that this vomit be included for analysis with the other body fluids, such a urine. In addition to monitoring fluid volume, it is important to securely contain any vomit or urine of a sick patient in order to minimize contamination and spread of disease. Therefore, there is a need to effectively seal the container once the fluid has been collected.

However, prior art devices for this purpose often fail to completely catch and hold the fluid which may miss entry into the container. This problem is particularly prevalent in situations where a patient is vomiting. Because vomiting is usually accompanied by large convulsive bodily movement, the patient generally finds it difficult to hold still. It may also be difficult for the patient to control arm and hand movement during actual regurgitation. Therefore, there is a likelihood that at least some of the regurgitated substance will miss any receptacle held by the person vomiting.

To facilitate collecting the body waste, collecting and disposing bags have been disclosed in the prior art which include openable funnels at their top to prevent spillage when receiving waste and internal “duck-bill” type valves within the bags. The “duck-bill” valves are depended upon as the principal means for sealing the bag's contents. In addition, score lines below the upper edge of the funnel permit upper portions of the funnel to be folded in overlapping relation for additional protection against spillage. However, the folded portions of the funnel do not form a leak proof seal and at best prevent large particles from escaping the bag.

The primary drawback of these known fluid collection bag designs is that the sealing means is insufficient either to prevent disease spread or spills in case of dropping or inverting the filled bag. In order to properly seal these bags, it is necessary to insert the filled bag into another container. Other bags have added the use of a seal strip comprising an interlocking vane and channel system to help the valve prevent spillage. Still others have incorporated bag-within-bag designs to prevent spillage.

These systems are typically complicated and expensive to manufacture. Therefore, there is a need for a device which provides for safe receipt, handling, and disposal of waste, such as vomit or urine. Such a device may be used when feeling ill or under medical observation, or in airplanes or other vehicles in which motion sickness may induce vomiting. While some of the requirements relating to sick patients do not apply to airplane passenger use, it is nonetheless important to collect any vomit as neatly as possible and to seal the collecting bag to prevent spillage in a moving airplane. In addition, providing a device which may be used in other ways, such as scooping up waste from the ground or other surfaces, would be an improvement over the prior art.

An improved device for receiving and disposing waste which addresses the problems of the prior art would be an important advance in the art. The present invention is directed to such a device which overcomes the disadvantages of the prior art.

### OBJECTS OF THE INVENTION

It is therefore an object of the present invention to provide a container for receiving body fluids which will protect the user from spillage.

Another object of the present invention is to provide a container which can help prevent the spreading of infectious or contagious diseases.

Another object of the present invention is to provide a disposable, collapsible bag which is easily sealed and disposed of without losing its waste contents.

A further object of the present invention is to provide a disposable, collapsible bag for receiving vomit and/or urine.

Yet another object of the present invention is to provide a simple and economical container for receiving waste such as vomit and urine.

Yet another object of the present invention is to provide a bag for the collection of waste with integral and substantially improved sealing means.

How these and other objects are accomplished will become apparent from the following descriptions and the drawings.

### SUMMARY OF THE INVENTION

This invention is an improved device for receiving and disposing of waste. The invention represents a significant advance over the state of the art by providing novel elements, including a foldable funnel with a sealing element pre-positioned to seal the funnel after receipt of waste.

In certain embodiments, the invention is a disposable device for receiving liquid waste and comprises a bag and a foldable funnel, the bag defining a cavity and having an aperture and the foldable funnel having a first opening and a second opening, the funnel bonded to the bag at the aperture such that the second opening is bounded by the bag, the funnel including a sealing element to seal the funnel such that the cavity is enclosed. When enclosed, the liquid waste cannot escape the device.

In certain embodiments, the second opening has a smaller diameter than the first opening. In such embodiments, the opening defines a bag circumference and the funnel has an outer surface defining a funnel circumference, the funnel circumference decreasing from the first opening to the second opening, the funnel bonded to the cavity at a position at which the funnel circumference is substantially equal to the bag circumference.

In certain embodiments, the funnel has an inner surface and the sealing element seals the inner surface. The sealing element may be an adhesive which, before use, is covered by a non-adhesive release liner, the liner being removed when the funnel is to be sealed to enclose the cavity.

In certain embodiments, the funnel includes a first crease line, a second crease line opposite the first crease line, a first funnel portion between the first and second crease lines, and a second funnel portion opposite the first funnel portion. In such embodiments, the sealing element is positioned on at least one of the funnel portions for sealing the funnel portions together to enclose the cavity.

In certain embodiments, at least a portion of the bag is transparent. Such transparency may allow a user to view the contents of the bag which, in some cases, may be beneficial. In certain embodiments the entire bag is transparent. In certain embodiments, the bag is flexible and the funnel is semi-rigid. In certain embodiments, the bag is bonded to the funnel with adhesive.

In certain embodiments, the invention is a disposable device for receiving liquid waste and the device comprises a flexible, substantially water-tight bag having a bottom and a wall extending from the bottom to an aperture, the bottom and wall defining a cavity for receiving waste; a funnel having an inner surface and an outer surface, the surfaces extending from a first opening to a second opening, the outer surface of the funnel bonded to the wall such that the second opening is in the cavity and the funnel extends from the bag such that the first opening is outside the cavity, the funnel having a first funnel portion and a second funnel portion, the funnel including a sealing element to seal the first and second funnel portions to one another to enclose the cavity.

In certain embodiments, the first and second funnel portions are separated by first and second crease lines. In such embodiments, the sealing element may be positioned on at least one of the funnel portions from the first crease line to the second crease line. The sealing element may be an adhesive and the adhesive may be covered by a non-adhesive release liner, the liner being removed when the funnel is to be sealed to enclose the cavity.

In certain embodiments, the sealing element seals the first funnel portion to the second funnel portions along the inner surface. In such embodiments, the sealing element may be an adhesive and the adhesive may be covered by a non-adhesive release liner, the liner being removed when the funnel is to be sealed to enclose the cavity.

In other embodiments, the invention is a method for collecting and disposing waste and the method comprises providing a disposable device having a bag and a funnel, the bag having a cavity and an aperture, the funnel having a first opening and a second opening, the funnel bonded to the bag such that the second opening is positioned in the cavity, the funnel including a sealing element; inserting waste into the bag through the funnel; folding the funnel such that the sealing element seals the funnel; and disposing the device. When the waste is vomit, the inserting action may be performed by placing the funnel near or around a mouth and vomiting into the bag through the funnel.

In certain embodiments the sealing element includes an adhesive, the adhesive is covered by a non-adhesive release liner, and the method further comprises removing the liner from the adhesive before the funnel is folded, the adhesive sealing the funnel upon folding.

In other embodiments, the invention is a method for catching and disposing a person's vomit and the method comprises providing a disposable device having a bag and a funnel, the bag having a cavity and an aperture, the funnel having a first opening and a second opening, the funnel bonded to the bag such that the second opening is positioned in the cavity, the funnel including a sealing element; placing funnel around the person's mouth; vomiting into the bag through the funnel; folding the funnel such that the sealing element seals the funnel; and disposing the device.

### BRIEF DESCRIPTION OF THE DRAWINGS

FIG. 1 is a perspective view of the device in accordance with an embodiment of the invention.

FIG. 2 is a front view of the device of FIG. 1.

FIG. 3 is a perspective view of the bag of the device in accordance with an embodiment of the invention.

FIG. 4 is a perspective view of the device with the bag of FIG. 3.

FIG. 5 is a perspective view of the device in accordance with an embodiment of the invention.

FIG. 6 is a perspective view of the device of FIG. 5 opened for use.

FIG. 7 is a perspective view of a person using the device in accordance with an embodiment of the invention.

### DETAILED DESCRIPTION OF PREFERRED EMBODIMENTS

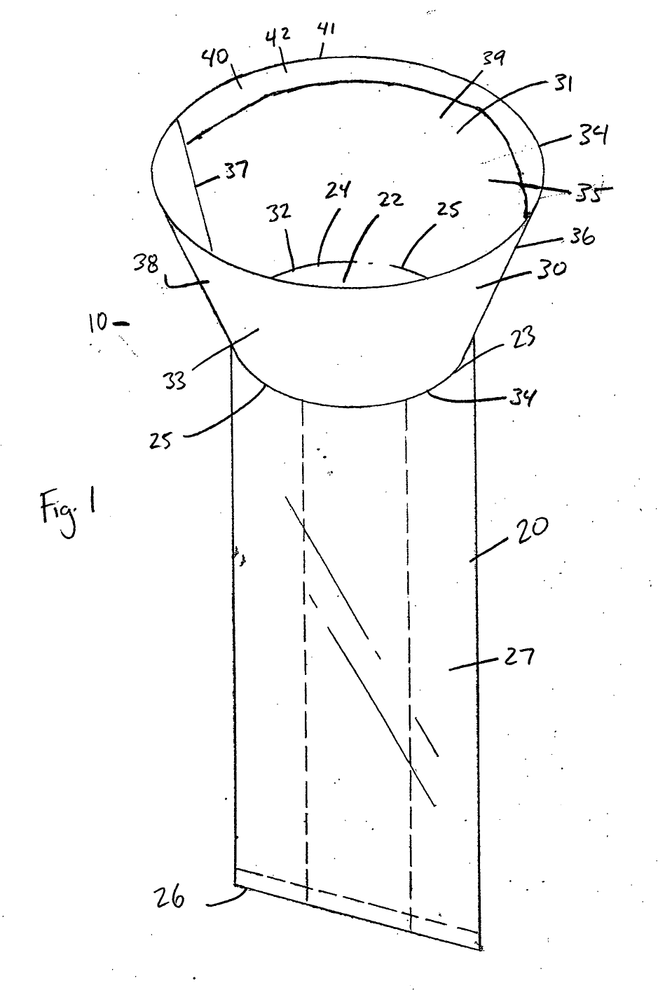

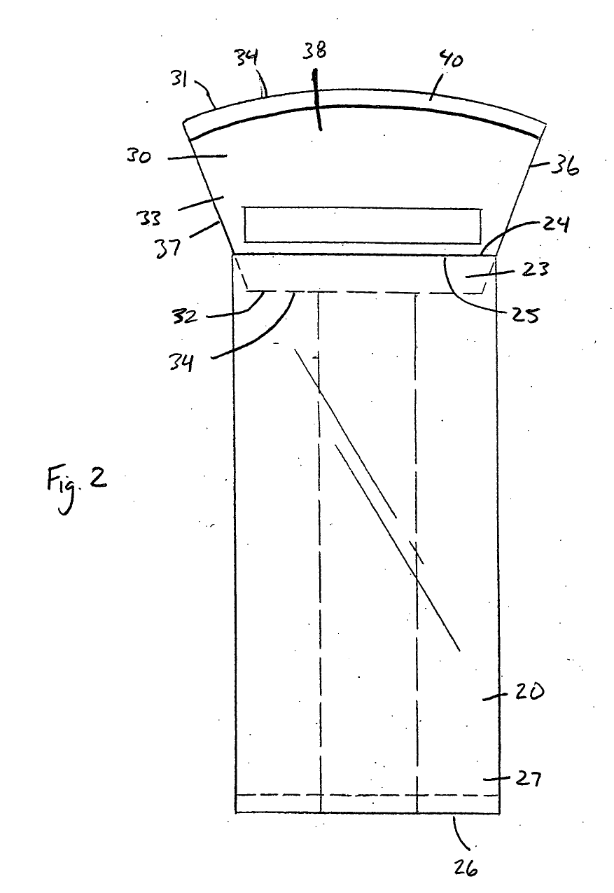

FIG. 1 is a perspective view, and FIG. 2 a front view, of a disposable device 10 for receiving and disposing of waste. Device 10 includes a bag 20 which has a bottom 26 and wall 27 extending from bottom 26 to an aperture 24. Wall 27 may be formed of planar portions, may be cylindrical, or may be of other known bag construction. Bag 10 is preferably flexible and water-tight. In certain embodiments, bag 10 is transparent to allow viewing of its contents, which may be necessary for medical evaluation. Aperture 24 leads to cavity 22 which is defined by bottom 26 and wall 27. Bag 10 has a bag circumference 25 which is shown as being substantially constant from bottom 26 to aperture 24.

Device 10 also includes a funnel 30 which extends from a first opening 31 to a second opening 32. Funnel 30 has a funnel circumference 34 which preferably decreases from first opening 31 to second opening 32. Funnel 30 includes an outer surface 33 and an inner surface 35. Funnel 30 has a first crease line 36 and a second crease line 37 with each crease line extending from first opening 31 to second opening 32 and allowing funnel 30 to be folded flat. Crease lines 36,37 divide funnel 30 into a first funnel portion 38 and a second funnel portion 39. As shown in FIG. 1, a sealing element 40 is positioned on second funnel portion 39 and allows sealing of second funnel portion 39 to first funnel portion 38 when funnel 30 is folded flat. Funnel 30 is preferably semi-rigid and can conform to a person's face when device 10 is used to receive vomit. In other embodiments, funnel 30 may be used to scoop waste from the ground or other surfaces.

As shown in FIGS. 1 and 2, funnel 30 extends into cavity 22 of bag 20. Funnel 30 and bag 20 are bonded to one another at a position at which bag circumference 25 and funnel circumference are substantially equal. Such bonding provides a water-tight seal between bag 20 and funnel 30 and may be provided by a strong adhesive positioned between the outer surface 33 of funnel 30 and the interior surface of bag 30, by a adhesive tape connected to the exterior surface of bag 20 and the outer surface 33 of funnel 30, or by other bonding means. Such bonding between bag 20 and funnel 30 requires that any material must pass through the openings 31,32 of funnel 30 to get into cavity 22 of bag 20.

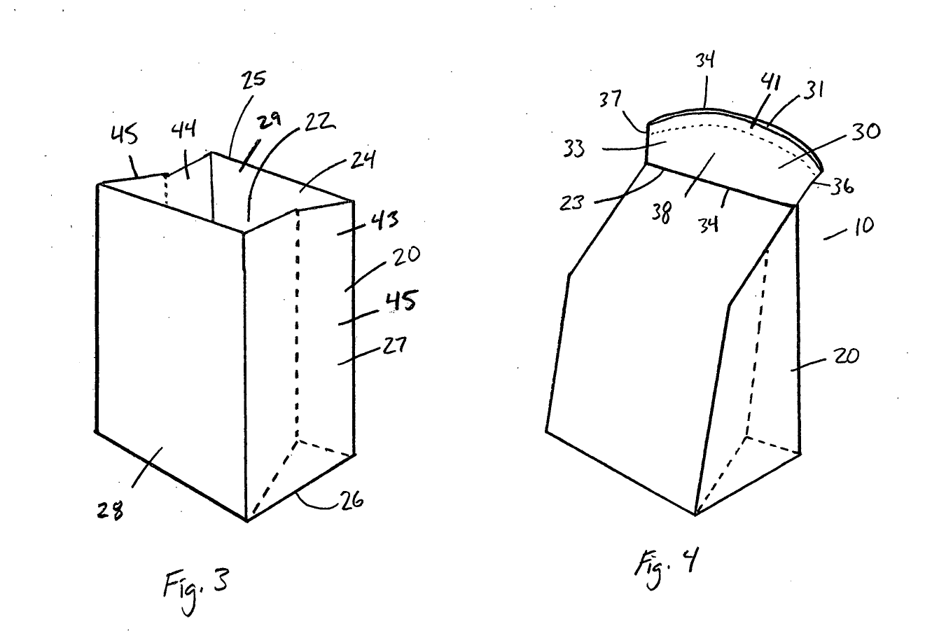

FIG. 3 shows a bag 20 for use in device 10. Bag 20 is of a typical construction which provides a large cavity 22 but allows bag 20 to be folded flat. Wall 27 includes opposite front portion 28 and rear portion 29 and opposite side portions 45. Side portions 45 include fold lines (shown by dashed lines) which facilitate folding bag 20 flat. When bag 20 is folded flat side portions 45 are folded toward cavity 22 such that side segments 43,44 are adjacent rear portion 29 and can be understood to affect the effective bag circumference 25. FIG. 4 shows a funnel 30 bonded to bag 20 at bond 23. Funnel 30 is positioned adjacent to rear portion 29 and side segments 43,44 fold against the edge sections of funnel 30 while front portion 28 contacts the middle section of funnel 30. Such a connection provides a pleat in bag 20 to allow for a large cavity 22. The entire aperture 24 of bag 20 is sealed and is water-tight so that no waste may unintentionally escape cavity 22. In FIG. 4, funnel 30 is folded flat and is sealed such that the inner surface 35 of first funnel portion 38 is completely adhered to the inner surface 35 of second funnel portion 39 by adhesive 41.

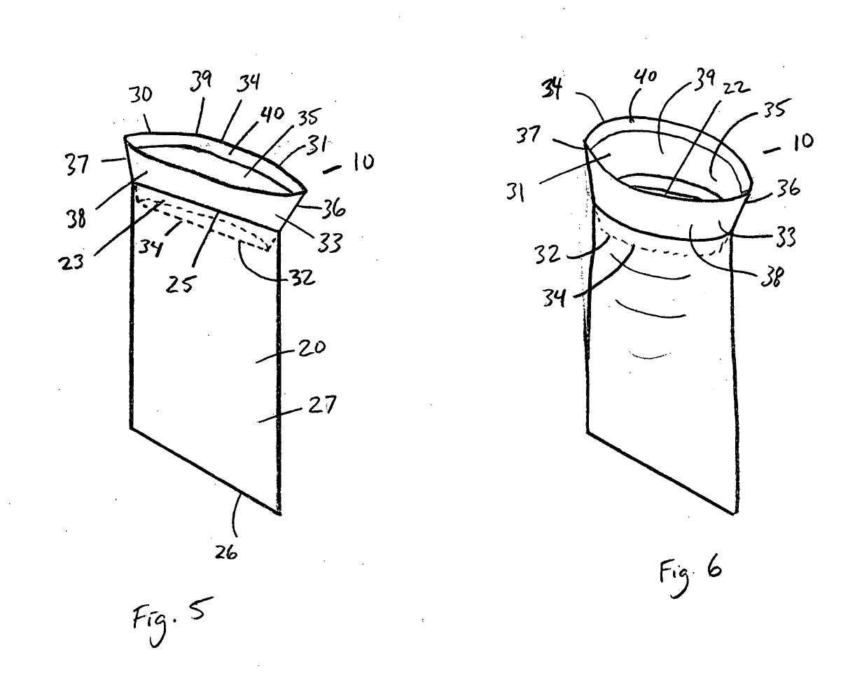

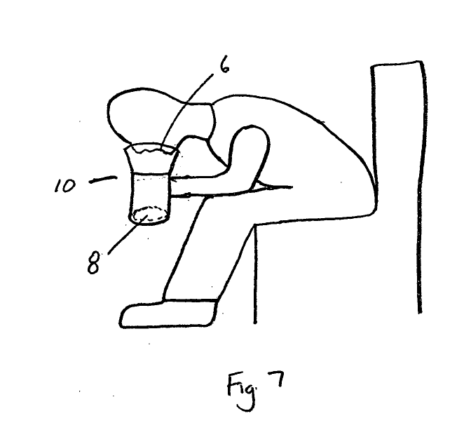

FIG. 5 shows device 10 having a differently designed bag 20. As shown, bag 20 includes a bottom 26 and a wall 27 comprising only a front and rear portion connected directly to one another. Wall 27 extends from bottom 26 to aperture 24 and has a substantially constant bag circumference 25. Funnel 30 extends into aperture 24 and is bonded to the front and rear portions of bag 20 at bond 23. Funnel 30 is shown slightly opened in FIG. 5 and more widely opened in FIG. 6.

FIG. 7 shows a person using device 10 to collect waste 8, specifically vomit. As shown, device 10 is opened is positioned around the person's mouth 6. Vomit 6 is received in the bag 20. When finished, funnel 30 is folded flat and sealed to prevent any waste 6 from escaping from device 10.

Thus, it should be apparent that there has been provided, in accordance with the present invention, a device for receiving and disposing of waste that fully satisfies the objectives and advantages set forth above.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A disposable device for receiving liquid waste, the device comprising:
a bag defining a cavity and having an aperture; and
a foldable funnel having a first opening and a second opening, the funnel bonded to the bag at the aperture such that the second opening is bounded by the bag, the funnel including a sealing element to seal the funnel such that the cavity is enclosed.

2. The device of claim 1 wherein the second opening has a smaller diameter than the first opening.

3. The device of claim 2 wherein the opening defines a bag circumference and the funnel has an outer surface defining a funnel circumference, the funnel circumference decreasing from the first opening to the second opening, the funnel bonded to the cavity at a position at which the funnel circumference is substantially equal to the bag circumference.

4. The device of claim 1 wherein the funnel has an inner surface and the sealing element seals the inner surface.

5. The device of claim 1 wherein the funnel includes a first crease line, a second crease line opposite the first crease line, a first funnel portion between the first and second crease lines, and a second funnel portion opposite the first funnel portion, the sealing element being positioned on at least one of the funnel portions for sealing the funnel portions together to enclose the cavity.

6. The device of claim 1 wherein the sealing element is an adhesive and wherein the adhesive is covered by a non-adhesive release liner, the liner being removed when the funnel is to be sealed to enclose the cavity.

7. The device of claim 1 wherein at least a portion of the bag is transparent.

8. The device of claim 1 wherein the bag is flexible and the funnel is semi-rigid.

9. The device of claim 1 wherein the bag is bonded to the funnel with adhesive.

10. A disposable device for receiving liquid waste, the device comprising:
a flexible, substantially water-tight bag having a bottom and a wall extending from the bottom to an aperture, the bottom and wall defining a cavity for receiving waste; and
a funnel having an inner surface and an outer surface, the surfaces extending from a first opening to a second opening, the outer surface of the funnel bonded to the wall such that the second opening is in the cavity and the funnel extends from the bag such that the first opening is outside the cavity, the funnel having a first funnel portion and a second funnel portion, the funnel including a sealing element to seal the first and second funnel portions to one another to enclose the cavity.

11. The device of claim 10 wherein the first and second funnel portions are separated by first and second crease lines.

12. The device of claim 11 wherein the sealing element is positioned on at least one of the funnel portions from the first crease line to the second crease line.

13. The device of claim 12 wherein the sealing element is an adhesive and wherein the adhesive is covered by a non-adhesive release liner, the liner being removed when the funnel is to be sealed to enclose the cavity.

14. The device of claim 10 wherein the sealing element seals the first funnel portion to the second funnel portions along the inner surface.

15. The device of claim 14 wherein the sealing element is an adhesive and wherein the adhesive is covered by a non-adhesive release liner, the liner being removed when the funnel is to be sealed to enclose the cavity.

16. The device of claim 10 wherein the second opening has a smaller diameter than the first opening.

17. The device of claim 10 wherein at least a portion of the bag is transparent.

18. A method for collecting and disposing waste, the method comprising:
providing a disposable device having a bag and a funnel, the bag having a cavity and an aperture, the funnel having a first opening and a second opening, the funnel bonded to the bag such that the second opening is positioned in the cavity, the funnel including a sealing element;
inserting waste into the bag through the funnel;
folding the funnel such that the sealing element seals the funnel; and
disposing the device.

19. The method of claim 18 wherein the waste is vomit and wherein the inserting action is performed by placing the funnel near a mouth and vomiting into the bag through the funnel.

20. The method of claim 18 wherein the sealing element includes an adhesive and wherein the adhesive is covered by a non-adhesive release liner, the method further comprising removing the liner from the adhesive before the funnel is folded, the adhesive sealing the funnel upon folding.
